(19) United States
(12) Patent Application Publication
(10) Pub. No.: US 2025/0205193 A1
(43) Pub. Date: Jun. 26, 2025

(54) THERAPEUTIC COMPOUND AND SALTS

(71) Applicant: Regents of the University of Minnesota, Minneapolis, MN (US)

(72) Inventors: Venkatram Reddy Mereddy, Minneapolis, MN (US); Zachary Scott Gardner, Minneapolis, MN (US); Tanner James Schumacher, Minneapolis, MN (US)

(73) Assignee: Regents of the University of Minnesota, Minneapolis, MN (US)

(21) Appl. No.: 18/846,873

(22) PCT Filed: Mar. 22, 2023
(86) PCT No.: PCT/US2023/015933

§ 371 Date: Sep. 13, 2024

Related U.S. Application Data
(60) Provisional application No. 63/322,443, filed on Mar. 22, 2022.

(51) Int. Cl. A61K 31/37; A61K 9/00; A61K 9/20; A61K 9/48; A61K 47/02; A61K 47/10; C07C 215/10; C07D 311/16

(52) U.S. Cl. CPC A61K 31/37; A61K 9/0019; A61K 9/2013; A61K 9/2018; A61K 9/2027; A61K 9/2054; A61K 9/2059; A61K 9/485; A61K 9/4858; A61K 9/4866; A61K 47/02; A61K 47/10; C07C 215/10; C07D 311/16

(57) ABSTRACT

The invention provides a compound of formula (I) or a salt thereof, as well as pharmaceutical compositions comprising a compound of formula (I) or a pharmaceutically acceptable salt thereof and methods of inhibiting an MPC and treating an MPC related disease or condition.

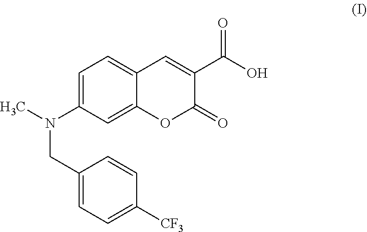 [SMILES: CN(Cc1ccc(C(F)(F)F)cc1)c1ccc2cc(C(=O)O)c(=O)oc2c1]

### CROSS-REFERENCE TO RELATED APPLICATION

This application claims priority to U.S. Provisional Application No. 63/322,443 that was filed on Mar. 22, 2022. The entire content of the application referenced above is hereby incorporated by reference herein.

### BACKGROUND

The recently characterized mitochondrial pyruvate carrier facilitates the transport of cytosolic pyruvate into the mitochondrial matrix. The MPC1 and MPC2 genes encode two obligate protein subunits of the MPC that form a heteroligomeric complex. Both proteins are required for activity as loss of one leads to destabilization and degradation of the MPC complex.

The MPC is found on the inner mitochondrial membrane and imports the metabolic end product of glycolysis, pyruvate, into the mitochondrial matrix for incorporation into intermediary metabolism in the citric acid cycle (TCA). Thus, MPC couples the two major energetic pathways, glycolysis and OxPhos, for energetic and biosynthetic needs of the rapidly proliferating cancer cells. Importantly, recent evidence suggests that highly oxidative cancer cell types exhibit increased levels of mitochondrial respiration and anabolic processes that drive cancer cell proliferation. Hence, targeting of MPC has high therapeutic potential for the treatment of cancer.

MCTs are members of the solute carrier 16-gene family consisting of 14 known isoforms. Of these, only MCTs 1-4 have been shown to elicit the proton-linked transport of monocarboxylates such as lactate, pyruvate, and some ketone bodies. MCT1 and MCT4 are centrally involved in glycolysis to efflux the end product lactate out of the tumor cells to avoid an apoptotic decrease in intracellular pH. They also play an active role in the influx of lactate from glycolytic cancer cells into the mitochondria of neighboring oxidative cancer cells for energy generation via OxPhos. Interestingly, the activity of MCT function is modulated by substrate accumulation in the cytosol and hence, reduced pyruvate uptake into the mitochondria via MPC inhibition has been shown to elicit feedback inhibition of MCTs. Hence, direct and/or feedback mediated inhibition of MCTs are important therapeutic targets for metabolism-directed cancer treatments.

Currently there is a need for agents that are direct or indirect inhibitors of MCTs. Such agents would be useful for the treatment of MCT related diseases and conditions (e.g., cancer, non-alcoholic steatohepatitis, diabetes, obesity, or chronic graft versus host disease).

### SUMMARY

Applicant has identified a compound that is an inhibitor of one or more MPC with potential to potently inhibit MCT1. The compound and its salts are useful for treating MPC and MCT related diseases and conditions (e.g., cancer, non-alcoholic steatohepatitis, diabetes, obesity, or chronic graft versus host disease). Accordingly, in one embodiment, the invention provides a compound of formula (I):

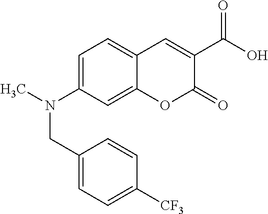 [SMILES: CN(Cc1ccc(C(F)(F)F)cc1)c1ccc2cc(C(=O)O)c(=O)oc2c1]

or a salt thereof.

The invention also provides a pharmaceutical composition comprising a compound of formula (I) or a pharmaceutically acceptable salt thereof, and a pharmaceutically acceptable excipient.

The invention also provides a method for inhibiting an MPC (in vivo or in vitro), comprising contacting the MPC with a compound of formula (I) or a salt thereof.

The invention also provides a method for treating an MPC related disease or condition (e.g., cancer, non-alcoholic steatohepatitis, diabetes, obesity, or chronic graft versus host disease) in an animal (e.g., a mammal such as a human) comprising administering a compound of formula (I) or a pharmaceutically acceptable salt thereof to the animal.

The invention also provides a compound of formula (I) or a pharmaceutically acceptable salt thereof for use in medical therapy.

The invention also provides a compound of formula (I) or a pharmaceutically acceptable salt thereof for the prophylactic or therapeutic treatment of an MPC related disease or condition (e.g., cancer, non-alcoholic steatohepatitis, diabetes, obesity, or chronic graft versus host disease).

The invention also provides the use of a compound of formula (I) or a pharmaceutically acceptable salt thereof to prepare a medicament for treating an MPC related disease or condition (e.g., cancer, non-alcoholic steatohepatitis, diabetes, obesity, or chronic graft versus host disease) in an animal (e.g. a mammal such as a human).

The invention also provides processes and intermediates disclosed herein that are useful for preparing a compound of formula (I) or a salt thereof.

### BRIEF DESCRIPTION OF THE DRAWINGS

FIG. 1 shows maximum tolerated dose data for the compound of formula (I) (Example 3).

FIG. 2 shows plasma concentration versus time graphs after oral and IV dosing of the compound of formula (I) (Example 4).

### DETAILED DESCRIPTION

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the objective is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention.

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” as used herein includes mammals. The term “mammal” refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient. In one embodiment, the patient is a human patient.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95% the absolute stereoisomer depicted. In another embodiment, the compound may be at least 99% the absolute stereoisomer depicted.

Processes for preparing compounds of formula (I) are provided as further embodiments of the invention and are illustrated by the following procedures in which the meanings of the generic radicals are as given above unless otherwise qualified.

A compound of formula (I) can be prepared as illustrated in the following Scheme.

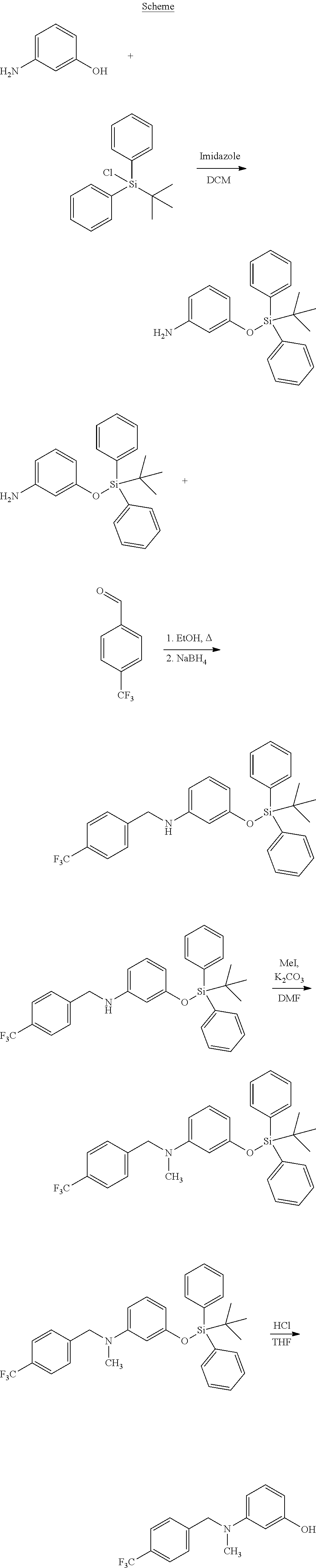

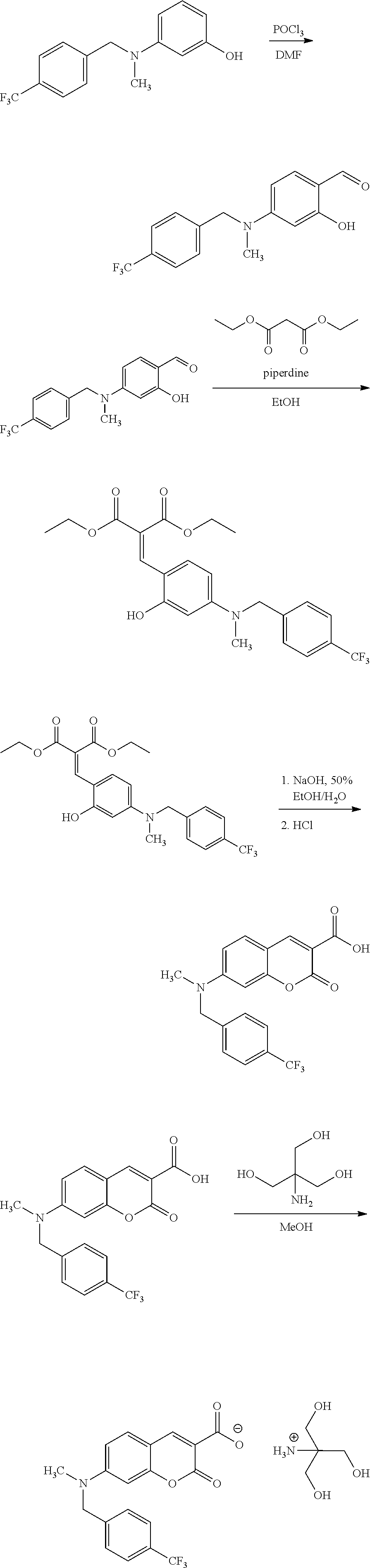

Intermediates useful for preparing a compound of formula (I) shown in the Scheme above represent an embodiment of the invention.

In cases where compounds are sufficiently basic or acidic, a salt of a compound of formula (I) can be useful as an intermediate for isolating or purifying a compound of formula (I). Additionally, administration of a compound of formula (I) as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula (I) can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gun tragacanth, acacia, corn starch or gelatin; excipients such as dicalcium phosphate; a disintegrating agent such as corn starch, potato starch, alginic acid and the like: a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula (I) to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula (I) can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for the treatment of cancer, non-alcoholic steatohepatitis, diabetes, obesity, or chronic graft versus host disease. Accordingly, in one embodiment the invention also provides a composition comprising a compound of formula (I), or a pharmaceutically acceptable salt thereof, at least one other therapeutic agent, and a pharmaceutically acceptable diluent or carrier. The invention also provides a kit comprising a compound of formula (I), or a pharmaceutically acceptable salt thereof, at least one other therapeutic agent, packaging material, and instructions for administering the compound of formula (I) or the pharmaceutically acceptable salt thereof and the other therapeutic agent or agents to an animal to treat cancer, non-alcoholic steatohepatitis, diabetes, obesity, or chronic graft versus host disease.

The ability of a compound of the invention to act as an agent for treating cancer, non-alcoholic steatohepatitis, diabetes, obesity, or chronic graft versus host disease may be determined using pharmacological models which are well known to the art, or using Test A described below.

The invention will now be illustrated by the following non-limiting Examples.

### EXAMPLES

### Example 1. Synthesis of 1,3-dihydroxy-2-(hydroxymethyl)propan-2-aminium 7-(methyl(4-(trifluoromethyl)benzyl)amino)-2-oxo-2H-chromene-3-carboxylate using tris base

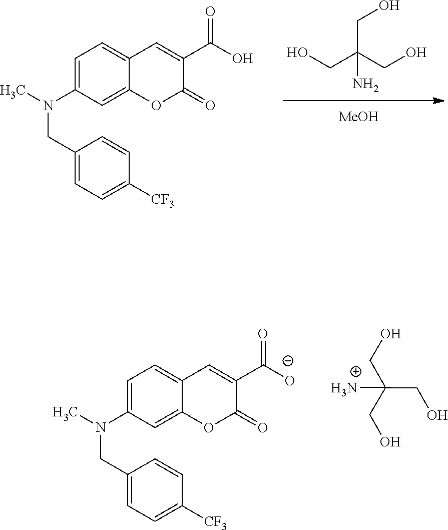 [SMILES: "MeOH" ; CN(Cc1ccc(C(F)(F)F)cc1)c1ccc2cc(C(=O)O)c(=O)oc2c1 ; NC(CO)(CO)CO ; CN(Cc1ccc(C(F)(F)F)cc1)c1ccc2cc(C(=O)[O-])c(=O)oc2c1 ; [NH3+]C(CO)(CO)CO]

The coumarin carboxylic acid (8 mmol) was dissolved in MeOH followed by addition of tris base (9.6 mmol). The reaction was allowed to stir for 3 hours after which it was concentrated under reduced pressure. Subsequent EtOAc washes removed excess tris base and afforded the title salt in 85% yield.

The starting coumarin carboxylic acid was prepared as follows.

### a. Synthesis of 3-((tert-butyldiphenylsilyl)oxy)aniline via silylation of 3-aminophenol

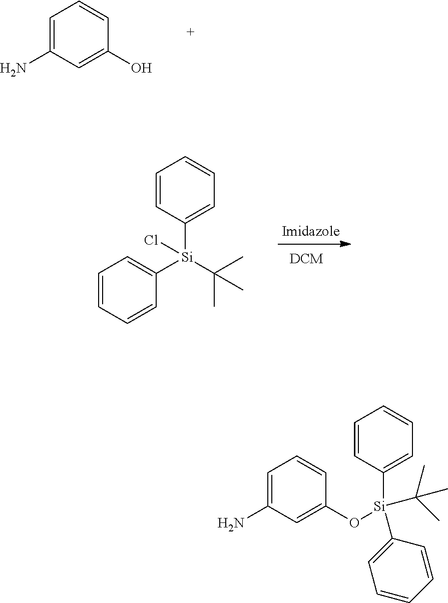 [SMILES: Nc1cccc(O)c1 ; CC(C)(C)[Si](Cl)(c1ccccc1)c1ccccc1 ; CC(C)(C)[Si](Oc1cccc(N)c1)(c1ccccc1)c1ccccc1]

3-Aminophenol (183 mmol) was dissolved in dichloromethane and brought to 0° C. Imidazole (366 mmol) was added slowly and the reaction was stirred for 5 minutes. A solution of tert-butylchlorodiphenylsilane (201.3 mmol) in dichloromethane was added dropwise. The reaction was brought to room temperature and progress was monitored via TLC (10% EtOAc/Hexanes). After 40 minutes or upon complete consumption of 3-aminophenol, the reaction was poured over 0.1 N aqueous HCl and extracted three times with dichloromethane (300 mL×3). The organic layer was dried with anhydrous magnesium sulfate and evaporated to yield subsequent red crude oil (95% yield).

### b. Synthesis of substituted N-benzyl-3-((tert-butyldiphenylsilyl)oxy)aniline

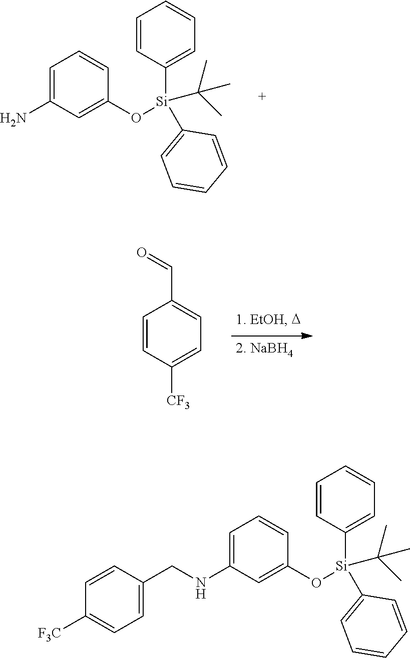 [SMILES: CC(C)(C)[Si](Oc1cccc(N)c1)(c1ccccc1)c1ccccc1 ; O=Cc1ccc(C(F)(F)F)cc1 ; CC(C)(C)[Si](Oc1cccc(NCc2ccc(C(F)(F)F)cc2)c1)(c1ccccc1)c1ccccc1]

The crude 3-((tert-butyldiphenylsilyl)oxy)aniline (29 mmol) was dissolved in anhydrous ethanol followed by substituted benzaldehyde (35 mmol) and refluxed. The subsequent imine formation was monitored via TLC (5% EtOAc/Hexanes). Upon complete consumption of the amine (6 hours), the reaction was brought to room temperature and sodium borohydride (14.5 mmol) was added in batches every 15 minutes. Upon complete reduction of the imine, the ethanol was evaporated under vacuum and stirred in a saturated sodium bicarbonate solution (300 mL). After stirring for 30 minutes, the mixture was extracted with diethyl ether (100 mL×3), dried with anhydrous magnesium sulfate and evaporated under vacuum to afford the resulting red crude oil (95% yield).

### c. Synthesis of substituted N-benzyl-3-((tert-butyldiphenylsilyl)oxy)-N-methylaniline

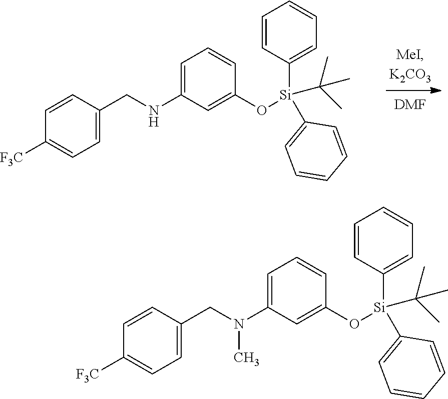

The N-benzyl-3-((tert-butyldiphenylsilyl)oxy)aniline crude (23 mmol) was dissolved in dimethylformamide followed by addition of potassium carbonate (69 mmol). The reaction was put on ice and methyl iodide (69 mmol) was added dropwise. The reaction was then brought to room temperature and reaction progress was monitored via TLC (5% EtOAc/Hexanes). The reaction was warmed at 40° C. Upon consumption of the starting material, the reaction was poured over water and extracted with diethyl ether (100 mL×3). The organic phase was dried with anhydrous magnesium sulfate and evaporated under vacuum to yield a crude amber oil winch was used in the next step without any further purification.

### d. Synthesis of substituted 3-(benzyl(methyl)amino)phenol

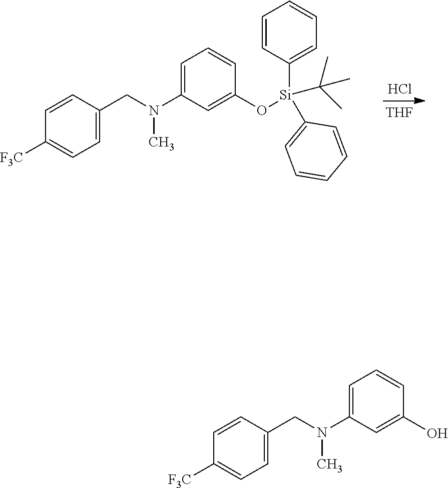 [SMILES: "THF" ; CN(Cc1ccc(C(F)(F)F)cc1)c1cccc(O[Si](c2ccccc2)(c2ccccc2)C(C)(C)C)c1 ; Cl ; CN(Cc1ccc(C(F)(F)F)cc1)c1cccc(O)c1]

The substituted N-benzyl-3-((tert-butyldiphenylsilyl)oxy)-N-methylaniline crude (23 mmol) was dissolved in THF, followed by the addition of HCl (230 mmol). The reaction was heated at 100° C. and the reaction progress was monitored via TLC (5% EtOAc/Hexanes). When the silylated starting material was entirely consumed, the THF was evaporated and the reaction was stirred in water (200 mL). The product was extracted with ethyl acetate (100 mL×3) and concentrated under vacuum to yield a red crude product which was used in the next step without any further purification.

### e. Synthesis of the substituted 4-(benzyl(methyl)amino)-2-hydroxybenzaldehyde

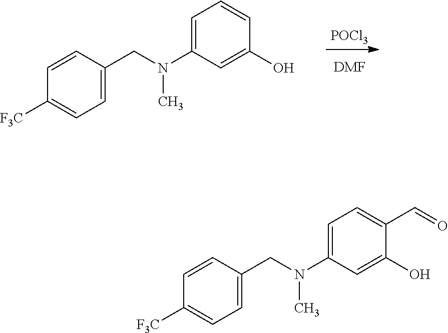

The substituted 3-(benzyl(methyl)amino)phenol (23 mmol) crude was dissolved in dimethylformamide (100 mL) and cooled on ice. To the reaction. POCl₃ (34.5 mmol) was added dropwise. The reaction was taken off ice and heated at 90° C. for 5 hours. Upon total consumption of the phenol, the reaction was poured over 400 mL of an ice-cold sodium carbonate (123 mmol) and extracted with ethyl acetate (100 mL×3). The organic phase was dried with magnesium sulfate and concentrated under vacuum to yield a blue crude oil which was used in the next step without any further purification.

### f. Synthesis of substituted diethyl 2-(4-(benzyl(methyl)amino)-2-hydroxy-benzylidene)malonate

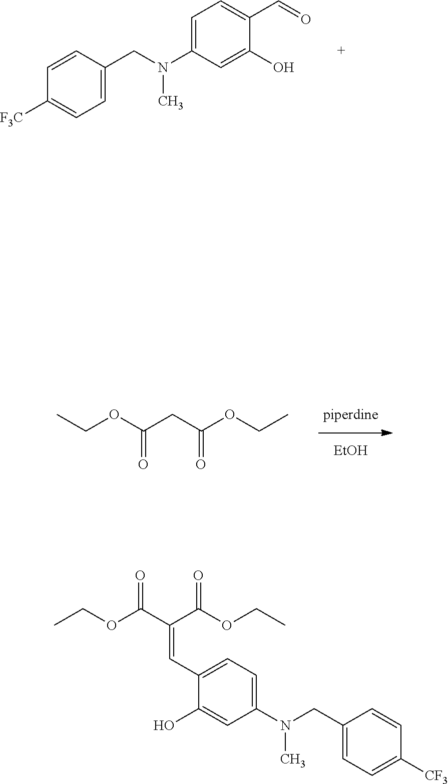

The substituted aldehyde (23 mmol) was dissolved in ethanol followed by addition of diethyl malonate (34.5 mmol). The reaction was cooled on ice followed by the addition of piperidine (27.6 mmol) and 4 drops of acetic acid. The reaction was removed from ice and refluxed for 12 hours. Reaction progress was monitored via TLC (20% EtOAc/Hexanes). Upon consumption of the aldehyde, the ethanol was evaporated, and the mixture was stirred in 300 mL of mildly acidic water for 20 minutes. Product was extracted with diethyl ether (100 mL×3), dried with anhydrous magnesium sulfate and concentrated under vacuum to give a vibrant red crude product which was used in the next step without any further purification.

### g. Synthesis of substituted 7-(benzyl(methyl)amino)-2-oxo-2H-chromene-3-carboxylic acid

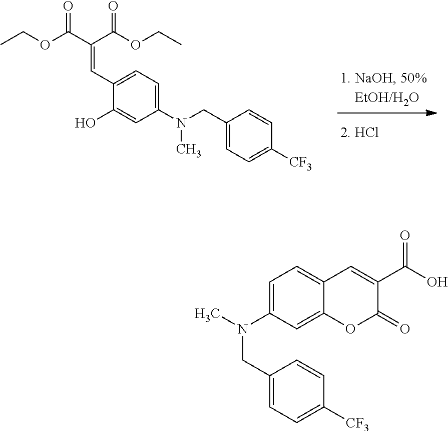 [SMILES: CCOC(=O)C(=Cc1ccc(N(C)Cc2ccc(C(F)(F)F)cc2)cc1O)C(=O)OCC ; CN(Cc1ccc(C(F)(F)F)cc1)c1ccc2cc(C(=O)O)c(=O)oc2c1]

The malonate crude (23 mmol) was dissolved in 200 mL ethanol and an aqueous sodium hydroxide solution (69 mmol) was added. This was heated to 50° C. and reaction progress was monitored via TLC (30% EtOAc/Hexanes). Upon consumption of the malonate, the ethanol was evaporated under vacuum to 50% volume and the mixture was poured over acidified brine. This was stirred for 2 hours, filtered and washed with cold water. The crude solid was recrystallized in ethanol to afford the coumarin carboxylic acid (50% yield).

### Example 2. Experimental Procedure for Biological Evaluation: Seahorse XFe96®-Based Respiratory Experiments in Permeabilized Cells for Pyruvate Driven Respiration

Permeabilized cell assays were performed using rPFO as described previously. 4T1 cells were seeded (20,000 cells/well) onto Seahorse XFe96 well plates and incubated overnight in growth media at 37° C. and 5% CO₂ for adherence. On the day of the assay, growth media was aspirated and replaced with mannitol/sucrose buffer (MAS; 70 mM sucrose, 220 mM mannitol, 10 mM potassium phosphate monobasic, 5 mM magnesium chloride, 2 mM HEPES, and 1 mM EGTA) after 3× rinse of growth media to remove serum and endogenous metabolic substrates, and incubated at 37° C. in a non-CO₂ incubator. Respective inhibitor and substrate milieus were prepared in MAS buffer for port injections A-D at 8×, 9×, 10×, and 11× the target cell concentrations to account for intrinsic dilution factor of in situ injections of each port. For pyruvate driven respiration experiments, test compound was injected in port A, followed by rPFO (1 nM) in port B, followed by respective substrate cocktails (FCCP stimulated) in port C, and rotenone and antimycin A (0.5 μM) in port D. Final substrate concentrations for specific tests were as follows: (5 mM pyruvate, 0.5 mM malate, 2 mM dichloroacetate (DCA); 10 mM glutamate, 2 mM DCA; 2 μM rotenone).

From these studies, it was found that the candidate compound inhibits pyruvate driven respiration with IC₅₀ values of 14.4=0.5 nM in 4T1 cells and 17.8±4.9 nM in 67NR cells. As a feedback mechanism, this candidate has excellent potential to inhibit MCT1 function.

### Example 3. Maximum Tolerated Dose Study

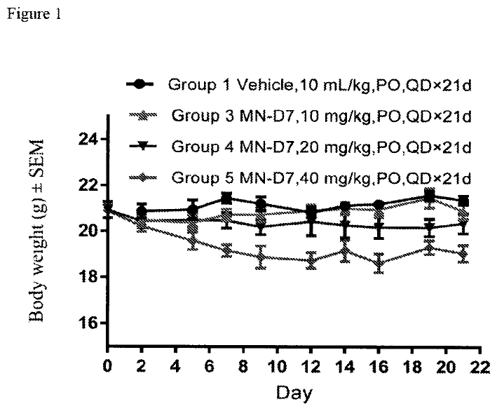

A maximum tolerated dose study was conducted in Balb-C mice. Results are shown in FIG. 1. This study indicated that the compound of formula (I) (MN-D7) was well tolerated at high doses (up to 40 mg/kg PO, q.d.) as evidenced by zero mortality, normal body weight gains and grooming patterns.

Additionally, a single dose acute toxicity study (40 mg/kg PO, q.d.) was carried out. This study showed that the compound of formula (I) did not cause any toxicity issues upon examination of internal organs of treated mice.

### Example 4. Pharmacokinetics and Oral Bioavailability

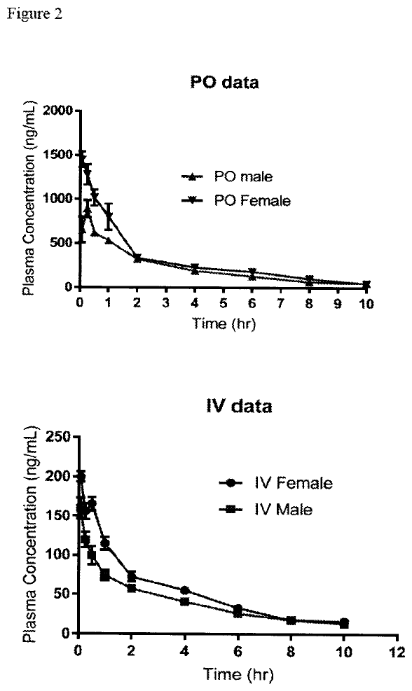

The pharmacokinetics and oral bioavailability of the compound of formula (I) were investigated in mouse following single intravenous and oral gavage. Results are shown in FIG. 2. After the IV dose, for the male animals the mean volume of distribution at steady state (Vss) was 10.24 L/kg. The mean blood clearance was 34.30 mL/min/kg. The mean terminal half-life was 3.65 hours; for the female animals the mean volume of distribution at steady state (Vss) was 6.92 L/kg. The mean blood clearance was 26.61 mL/min/kg. The mean terminal half-life was 3.15 hours (table 2).

After the PO dose, for the male animals the mean oral bioavailability was estimated to be about 124.96%. The mean terminal half-life was 4.04 hours; for the female animals, the mean oral bioavailability was estimated to be about 123.84%. The mean terminal half-life was 4.41 hours. Data is shown in the following table. The compound of formula (I) is identified as MN-D7.

TABLE 2
PK parameters

| | | | | HL_Lambda | Tmax | Cmax | AUClast | AUCINF_o [symbol] |
|---|---|---|---|---|---|---|---|---|
| G1 | | Administration | Dose Leve [symbol] | T_{1/2} | Tₘₐₓ | Cₘₐₓ | AUC₍₀₋ₜ₎ | AUC_{(0-∞)} |
| Animal No. | Analyte | Route | mg/kg | h | h | ng/mL | h*ng/mL | h*ng/mL |
| G1-Male | MN-D7 | IV | 1.0 | 3.65 | 0.08 | 160.07 | 413.43 | 485.94 |
| G1-Female | | IV | 1.0 | 3.15 | 0.08 | 199.82 | 551.74 | 626.43 |

| | MRTlast | MRTINF_ [symbol] | C0 | Vss_obs | Vz_obs | Cl_obs |
|---|---|---|---|---|---|---|
| G1 | MRT₍₀₋ₜ₎ | MRT_{(0-∞)} | C₀ | Vₛₛ | V_{z} | Cl |
| Animal No. | h | h | ng/mL | mL/kg | mL/kg | mL/h/kg |
| G1-Male | 3.17 | 4.97 | 185.14 | 10235.23 | 10835.40 | 2057.88 |
| G1-Female | 2.95 | 4.34 | 226.05 | 6920.88 | 7261.08 | 1596.36 |

| G2 | | Administration | Dose Leve [symbol] | T_{1/2} | Tₘₐₓ | Cₘₐₓ | AUC₍₀₋ₜ₎ | AUC_{(0-∞)} | MRT₍₀₋ₜ₎ | MRT_{(0-∞)} | F |
|---|---|---|---|---|---|---|---|---|---|---|---|
| Animal No. | Analyte | Route | mg/kg | h | h | ng/mL | h*ng/mL | h*ng/mL | h | h | % |
| G2-Male | MN-D7 | PO | 5.00 | 4.04 | 0.25 | 889.99 | 2583.14 | 2609.19 | 4.07 | 4.33 | 124.96 |
| G2-Female | | PO | 5.00 | 4.41 | 0.08 | 1449.47 | 3416.23 | 3483.13 | 3.88 | 4.39 | 123.84 |

[symbol]

indicates data missing or illegible when filed

### Example 5

The following illustrate representative pharmaceutical dosage forms, containing a compound of formula (I) (‘Compound X’), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X= (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X= (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula (I): 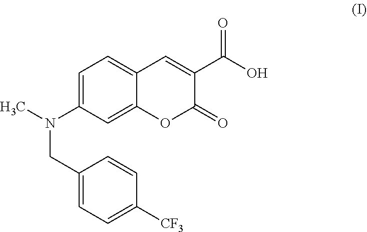 [SMILES: CN(Cc1ccc(C(F)(F)F)cc1)c1ccc2cc(C(=O)O)c(=O)oc2c1]
or a salt thereof.

2. The salt of claim 1, which is a pharmaceutically acceptable salt.

3. The salt of claim 1, which is

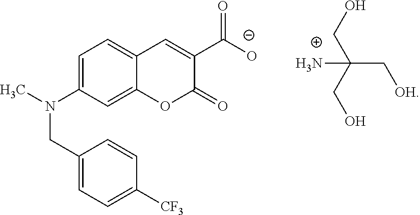 [SMILES: CN(Cc1ccc(C(F)(F)F)cc1)c1ccc2cc(C(=O)[O-])c(=O)oc2c1 ; [NH3+]C(CO)(CO)CO]

4. A pharmaceutical composition comprising a compound of formula (I) as described in claim 1 or a pharmaceutically acceptable salt thereof, and a pharmaceutically acceptable excipient.

5. A method for inhibiting an MPC (in vivo or in vitro), comprising contacting the MPC with a compound of formula (I) or a salt as described in any one of claims 1-3.

6. A method for treating an MPC related disease or condition (e.g., cancer, non-alcoholic steatohepatitis, diabetes, obesity, or chronic graft versus host disease) in an animal (e.g., a mammal such as a human) comprising administering a compound of formula (I) as described in claim 1 or a pharmaceutically acceptable salt thereof to the animal.

7. A compound of formula (I) as described in claim 1 or a pharmaceutically acceptable salt thereof for use in medical therapy.

8. A compound of formula (I) as described in claim 1 or a pharmaceutically acceptable salt thereof for the prophylactic or therapeutic treatment of an MPC related disease or condition (e.g., cancer, non-alcoholic steatohepatitis, diabetes, obesity, or chronic graft versus host disease).

9. The use of a compound of formula (I) as described in claim 1 or a pharmaceutically acceptable salt thereof to prepare a medicament for treating an MPC related disease or condition (e.g., cancer, non-alcoholic steatohepatitis, diabetes, obesity, or chronic graft versus host disease) in an animal (e.g. a mammal such as a human).
